(19) United States
(12) Patent Application Publication
(10) Pub. No.: US 2023/0416777 A1
(43) Pub. Date: Dec. 28, 2023

(54) MOLONEY MURINE LEUKEMIA VIRUS-BASED SELF-INACTIVATING VECTOR AND APPLICATIONS THEREOF

(71) Applicant: YUNZHOU BIOSCIENCES (GUANGZHOU) CO., INC., Guangzhou, Guangdong (JP)

(72) Inventors: Jinxiu SHI, Guangzhou, Guangdong (CN); Weineng MENG, Guangzhou, Guangdong (CN); Yan LUO, Guangzhou, Guangdong (CN); Yingjun LIN, Guangzhou, Guangdong (CN); Shengzhi LIN, Guangzhou, Guangdong (CN)

(73) Assignee: YUNZHOU BIOSCIENCES (GUANGZHOU) CO., LTD., Guangzhou, Guangdong (CN)

(21) Appl. No.: 18/038,976

(22) PCT Filed: Jun. 15, 2021
(86) PCT No.: PCT/CN2021/100014

§ 371 Date: May 25, 2023

(30) Foreign Application Priority Data
Nov. 25, 2020 (CN) 202011343883.8

(51) Int. Cl. C12N 15/86; C12N 15/65; A61K 48/00

(52) U.S. Cl. CPC C12N 15/86; C12N 15/65; A61K 48/0008; C12N 2800/107; C12N 2820/55; C12N 2830/48; C12N 2830/50; C12N 2740/13043; C12N 2740/13052

(57) ABSTRACT

Provided are a Moloney murine leukemia virus-based self-inactivating vector and applications thereof. The self-inactivating vector comprises: 5′LTR, a target expression gene or multiple cloning sites used for inserting the target expression gene, 3′LTR, and a polyadenylated nucleic acid fragment, where the nucleic acid fragment between the Pvu II enzyme cutting site and the Sac I enzyme cutting site of the U3 region of 5′LTR is replaced by a CMV enhancer and a CMV promoter connected to the CMV enhancer, the nucleic acid fragment between the Pvu II enzyme cutting site and the Sac I enzyme cutting site of the U3 region of 3′LTR is deleted, and the polyadenylated nucleic acid fragment is located at the 3′-terminus of 3′LTR. The self-inactivating vector, serving as a vector for a gene therapy, provides increased safeness and a high virus titer, and allows the high-efficiency expression of a target gene.

### FIELD

The present invention relates to the technical field of genetic engineering, in particular to a moloney murine leukemia virus-based self-inactivating vector and uses thereof.

### BACKGROUND

Moloney murine leukemia virus (MMLV) belongs to the γ-retrovirus family. Moloney murine leukemia virus vector is a gene expression system that can efficiently integrate exogenous genes into mammal cell genome randomly and stably. The integration mechanism is that, when the virus transduces the target cell, the glycoprotein on the surface of the virus binds to the receptor on the cell surface, mediating the entry of the viral RNA into the host cell. The viral RNA is further reversely transcribed into DNA with the help of its own reverse transcriptase. Subsequently, the viral genome located between two long terminal repeats (LTRs) is randomly integrated into the host cell genome. MMLV vector has the advantages of wide range of host, high transduction efficiency, and ability of achieving significant long-lasting expression of exogenous genes. MMLV vector has been used as a gene therapy vector in the treatment of children with X-linked severe combined immunodeficiency (SCID-X1). Within 2 to 6 years after the treatment, although most patients showed a good therapeutic effect, leukemia occurred in a small number of patients, which may be due to the retention of two identical wild-type LTRs on the traditional MMLV retrovirus vector (5′LTR and 3′LTR). When MMLV is randomly integrated into a mammalian genome, the promoter and enhancer contained in the LTR may activate the expression of a host gene flanking the integration site, and when the integration occurs near a proto-oncogene, the proto-oncogene may be activated, thereby causing tumorigenesis. Moreover, the viral promoter carried by LTR will compete with the internal promoter, which may interfere the improvement of virus titer and the efficient expression of exogenous genes. In addition, it is possible that two highly homologous LTRs undergo homologous recombination, which may affect the stability of the vector.

In order to solve the problem of easy activation of proto-oncogenes in the process of MMLV gene therapy, various MMLV self-inactivating (SIN) vectors have been developed. In all of these vectors, part of the promoter and enhancer sequences of the virus in U3 region of 3′LTR have been deleted (i.e., the sequence between PvuII enzyme recognition site and XbaI enzyme recognition site has been deleted), which not only reduces the risk of gene upregulation near the insertion site, but also increases the autonomy of the internal promoter. However, studies have found that for gene therapy, the safety of these SIN vectors needs to be further improved.

### SUMMARY

Based on the above, it is necessary to provide a moloney murine leukemia virus-based self-inactivating vector having improved safety and more suitable for gene therapy.

Provided is a Moloney murine leukemia virus-based self-inactivating vector comprising:
5′LTR, wherein a nucleic acid fragment between Pvu II enzyme recognition site and Sac I enzyme recognition site in U3 region of the 5′LTR of the self-inactivating vector is replaced by CMV enhancer and CMV promoter connected to the CMV enhancer;
a target gene or a multiple cloning site for insertion of the target gene; and
3′LTR, wherein a nucleic acid fragment between a PvuII enzyme recognition site and a Sac I enzyme recognition site in U3 region of the 3′LTR of the self-inactivating vector is deleted, and a polyadenylation signal sequence is located at the 3′-terminal of the 3′LTR of the self-inactivating vector.

In the Moloney murine leukemia virus-based self-inactivating vector of the present invention, a nucleic acid fragment between PvuII enzyme recognition site and SacI enzyme recognition site in U3 region of 5′LTR is deleted, and a nucleic acid fragment between PvuII recognition site and SacI enzyme recognition site in U3 region of 3′LTR is deleted, so that there is no CAAT box in U3 region of 5′LTR and U3 region of 3′LTR of the Moloney murine leukemia virus-based self-inactivating vector, thereby reducing the risk of insertional activation of the Moloney murine leukemia virus-based self-inactivating vector, and improving the safety of the self-inactivating vector. In addition, in the Moloney murine leukemia virus-based self-inactivating vector, CMV enhancer and CMV promoter are inserted at the 5′ end of SacI enzyme recognition site in U3 region of 5′LTR, and a polyadenylation signal sequence is added to the 3′ end of U3 region of 3′LTR, increasing the titer of the Moloney murine leukemia virus-based self-inactivating vector. Therefore, the Moloney murine leukemia virus-based self-inactivating vector of the present invention is a self-inactivating vector with high virus titer and no risk of insertional activation.

In an embodiment, the nucleotide sequence of the 5′LTR is set forth in SEQ ID NO: 1; and/or, the nucleotide sequence of the 3′LTR is set forth in SEQ ID NO: 2.

In an embodiment, the polyadenylation signal sequence is derived from Simian virus 40, and the nucleotide sequence of the polyadenylation signal sequence is set forth in SEQ ID NO: 3.

In an embodiment, the self-inactivating vector further comprises a post-transcriptional regulatory element, wherein the post-transcriptional regulatory element is located close to the 5′ terminal of the 3′LTR, and at the 3′ terminal of the target gene or the multiple cloning site; optionally, the post-transcriptional regulatory element is oPRE.

In an embodiment, the self-inactivating vector further comprises a replication origin derived from bacteria and a resistance gene as a marker.

In an embodiment, the replication origin is pUC ori;
and the resistance gene is a gene encoding a protein with resistance to a drug selected from the group consisting of kanamycin, zeocin, actinomycin, ampicillin, gentamicin, tetracycline, chloramphenicol, penicillin and a combination thereof.

In an embodiment, the self-inactivating vector further comprises a reporter gene.

Provided is a retrovirus packaging system comprising at least two plasmids, wherein one of the plasmids is the Moloney murine leukemia virus-based self-inactivating vector comprising a target gene.

Provided is a virus particle generated by transfecting a host cell with the retrovirus packaging system.

Provided is a gene therapy drug comprising the virus particle and a pharmaceutically acceptable excipient.

### BRIEF DESCRIPTION OF DRAWINGS

FIG. 1 is a schematic diagram of the structure of the MMLV self-inactivating vector 4C in Example 1;

FIG. 2 is a schematic diagram of the structure of the MMLV self-inactivating vector 4D in Example 1;

FIG. 3 is a schematic diagram of the structure of the MMLV self-inactivating vector oPRE-4C in Example 1;

FIG. 4 is a schematic diagram of the structure of the MMLV self-inactivating vector oPRE-4D in Example 1; and

FIG. 5 is a fluorescence image of HEK293T cells infected by each packaged MMLV self-inactivating vector in Example 1.

### DETAILED DESCRIPTION

In order to facilitate the understanding of the present invention, the following will describe the present invention more fully. However, the present invention may be realized in many different forms and is not limited to the examples described herein. On the contrary, these examples are provided to make the disclosed content of the present invention more thorough and complete.

Unless otherwise defined, all technical and scientific terms used herein have the same meaning as commonly understood by those of ordinary skill in the technical field of the present invention. The terms used in the specification of the present invention are for the purpose of describing specific examples only, and are not intended to limit the present invention.

In an embodiment, the present invention provides a Moloney murine leukemia virus-based self-inactivating vector comprising 5′LTR, a target gene or a multiple cloning site for inserting a target gene, 3′LTR and a polyadenylation signal sequence. A nucleic acid fragment between PvuII enzyme recognition site and SacI enzyme recognition site in U3 region of 5′LTR of the self-inactivating vector is replaced by CMV enhancer and CMV promoter connected to the CMV enhancer. A nucleic acid fragment between PvuII enzyme recognition site and SacI enzyme recognition site in U3 region of 3′LTR of the self-inactivating vector is deleted. The polyadenylation signal sequence is located at 3′ end of 3′LTR of the self-inactivating vector.

During the research, it has been found that traditional MMLV self-inactivating vector still comprises CAAT box and partial promoter sequence that denote the characteristic of traditional promoter sequences before SacI enzyme recognition site in U3 region of 3′LTR (i.e., between PvuII enzyme recognition site and SacI enzyme recognition site), indicating that there is still a risk of insertional upregulation. This factor needs to be taken into account when assessing the risk of tumorigenesis in gene therapy strategy using MMLV-based self-inactivating vector. After the ideal MMLV self-inactivating vector with high safety is integrated into the genome, there should not be any activity of promoter/enhancer derived from virus in LTR, and the expression of the exogenous target gene only depends on the internal promoter. Therefore, in order to further improve the biological safety of the MMLV self-inactivating vector, eliminate the influence of the potential promoter/enhancer activity in the MMLV self-inactivating vector, and enable the target gene to be expressed efficiently, the MMLV self-inactivating vector is further modified. For U3 region of 5′LTR, a nucleic acid fragment between PvuII enzyme recognition site and SacI enzyme recognition site in U3 region is deleted. However, the deletion of this region may seriously weaken the transcription ability of the viral genome, resulting in a sharp drop in virus titer. In order to overcome this defect, RSV promoter, SV40 enhancer+RSV promoter or CMV enhancer+CMV promoter are added at the 5′ end of SacI enzyme recognition site. For U3 region of 3′LTR, a nucleic acid fragment between PvuII enzyme recognition site and XbaI enzyme recognition site in U3 region is deleted, and a sequence between XbaI site and SacI site in U3 region is further deleted. In addition, in order to further strengthen the ability of post-transcriptional processing of viral genome RNA and increase virus titer, a polyadenylation signal sequence is added to the 3′ end of 3′LTR as an element to enhance RNA stability.

In the present embodiments, a nucleic acid fragment between PvuII enzyme recognition site and SacI enzyme recognition site of U3 region of 5′LTR of the Moloney murine leukemia virus-based self-inactivating vector is replaced with CMV enhancer and CMV promoter connected to CMV enhancer. Particularly, the nucleotide sequence of the 5′LTR of the Moloney murine leukemia virus-based self-inactivating vector is set forth in SEQ ID NO: 1; and/or, the nucleotide sequence of the 3′LTR of the Moloney murine leukemia virus-based self-inactivating vector is set forth in SEQ ID NO: 2. Particularly, the polyadenylation signal sequence is derived from Simian virus 40, and the nucleotide sequence of the polyadenylation signal sequence is set forth in SEQ ID NO: 3.

In the present embodiment, PvuII enzyme recognition site and SacI enzyme recognition site in U3 region of 5′LTR, and PvuII enzyme recognition site and SacI enzyme recognition site in U3 region of 3′LTR are not deleted. In some embodiments, at least one of PvuII enzyme recognition site and SacI enzyme recognition site in U3 region of 5′LTR may be deleted, and at least one of PvuII enzyme recognition site and SacI enzyme recognition site in U3 region of 3′LTR may be deleted.

In some embodiments, the above Moloney murine leukemia virus-based self-inactivating vector further comprises a post-transcriptional regulatory element. The post-transcriptional regulatory element is used to increase the expression level of exogenous genes (i.e., target genes). The post-transcriptional regulatory element is located between 3′LTR and the target gene or the multiple cloning site for insertion of the target gene, i.e., between the 5′ end of 3′LTR and the 3′ end of the target gene or the multiple cloning site. Further, the post-transcriptional regulatory element is oPRE. oPRE is optimized from WPRE, which can enhance the processing of the transcriptional product 3′RNA, thereby significantly increasing the expression level of exogenous genes. Compared with WPRE, the promoter and coding region of WHVX are removed from oPRE, and the ATGs in 4 ORFs of WPRE that may encode proteins with more than 25 amino acids are mutated, thereby overcoming the potential risk of tumorigenesis of WPRE.

In some embodiments, the Moloney murine leukemia virus-based self-inactivating vector of the present invention further comprises a replication origin derived from bacteria and a resistance gene as a marker. The replication origin derived from bacteria and resistance gene are used to initiate the transcription and expression of the resistance gene, which is used to screen the correct vectors after construction. Optionally, the replication origin is pUC ori. In another embodiment, the replication origin is not limited to pUC ori, and may be other replication origins derived from bacteria. Optionally, the resistance gene is a gene encoding a protein with resistance to a drug selected from the group consisting of kanamycin, zeocin, actinomycin, ampicillin, gentamicin, tetracycline, chloramphenicol, penicillin and a combination thereof.

In some embodiments, the Moloney murine leukemia virus-based self-inactivating vector of the present invention further comprises a reporter gene. Particularly, the reporter gene is a fluorescent reporter gene. More particularly, the fluorescent reporter gene is EGFP. In another embodiment, the reporter gene is not limited to the above, and may be selected according to specific needs.

Due to the deletion of the nucleic acid fragment between PvuII enzyme recognition site and SacI enzyme recognition site in U3 region of 5′LTR and the deletion of the nucleic acid fragment between PvuII enzyme recognition site and SacI enzyme recognition site in U3 region of 3′LTR in the Moloney murine leukemia virus-based self-inactivating vector, there is no CAAT box in U3 regions of 5′LTR and 3′LTR of the Moloney murine leukemia virus-based self-inactivating vector, thereby reducing the risk of insertional activation of the Moloney murine leukemia virus-based self-inactivating vector, and improving the safety of the self-inactivating vector. In addition, in the above Moloney murine leukemia virus-based self-inactivating vector, CMV enhancer and CMV promoter are inserted in U3 region of 5′LTR, a polyadenylation signal sequence is added to the 3′ end of U3 region of 3′LTR, and a post-transcriptional regulatory element is added at the 3′ end of the target gene, so that the Moloney murine leukemia virus-based self-inactivating vector has high virus titer, and the expression level of the exogenous gene inserted in the self-inactivating vector is high. Therefore, the above Moloney murine leukemia virus-based self-inactivating vector is a self-inactivating vector with high virus titer and no risk of insertional activation, which can increase the expression level of exogenous genes.

An embodiment of the present invention further provides a retrovirus packaging system comprising at least two plasmids, capable of producing retrovirus particles with infection ability (infect once) but no replication ability, wherein one of the plasmids is the Moloney murine leukemia virus-based self-inactivating vector, which contains the genetic information required for packaging, transfection and stable integration into the host cell, and serves to integrate the target gene into the genome of the host cell.

Optionally, the above retrovirus packaging system is a three-plasmid system, comprising the Moloney murine leukemia virus-based self-inactivating vector, an envelope protein expression plasmid and gag-pol expression plasmid. The gag-pol expression plasmid contains gag gene and pol gene. The gag gene encodes a nucleocapsid protein, an inner membrane protein and a capsid protein. The pol gene encodes a polymerase precursor protein that is cleaved to generate protease, integrase, reverse transcriptase, and ribonuclease H. The RNA of the Moloney murine leukemia virus forms a hairpin structure, i.e. the so-called stem-loop of R region, between the cap and poly(A) signal, and promotes the accumulation of unspliced transcripts in the cytoplasm, so that the viral genomic RNA, capsid protein, reverse transcriptase, integrase, envelope protein and the like are assembled into pseudovirion particles. In another embodiment, the above retrovirus packaging system may be a two-plasmid system packaged using a cell line stably expressing gag-pol.

The above retrovirus packaging system comprises the above Moloney murine leukemia virus-based self-inactivating vector, and can be used to prepare virus particles efficiently expressing exogenous genes with high safety.

An embodiment of the present invention further provides a virus particle, which is generated by transfecting a host cell with the above retrovirus packaging system. The virus particle generated by the retrovirus packaging system has no risk of insertional activation and can express the target gene efficiently.

In addition, an embodiment of the present invention further provides use of the virus particle in the manufacture of a medicament for gene therapy.

An embodiment of the present invention further provides a medicament for gene therapy, which comprises the virus particle and a pharmaceutically acceptable excipient.

The medicament for gene therapy comprises the above virus particle, which compensates or corrects the diseases caused by gene defects or gene abnormalities by introducing a normal or therapeutic target gene or nucleic acid molecule into a target cell, so as to achieve the therapeutic effect. The above medicament for gene therapy comprises the above virus particle, and has high safety.

### EXAMPLES

The following will be described in detail in conjunction with specific examples. Unless otherwise specified, the following examples do not include other components except unavoidable impurities. Unless otherwise specified, the reagents and instruments used in the examples are all conventional choices in the art. In the examples, the experimental methods for which specific conditions are not indicated are implemented according to conventional conditions, for example, the conditions described in literature and books or the method recommended by manufacturers.

### Example 1

1. Construction of MMLV Self-Inactivating Vectors with Various 5′LTR and 3′LTR

Due to the large number of vectors to be constructed, in order to quickly determine the LTR combination manners of different vectors, 5′LTR and 3′LTR of each vector were numbered. 5′LTRs were sequentially numbered as 1, 2, 3 and 4, which had corresponding structural features as follows:
1 corresponding to the 5′LTR of wild-type MMLV, as a control;
2 corresponding to the replacement of the sequence between PvuII enzyme recognition site and SacI enzyme recognition site in wild-type 5′LTR with RSV promoter;
3 corresponding to the replacement of the sequence between PvuII enzyme recognition site and SacI enzyme recognition site in wild-type 5′LTR with SV40_En-RSV promoter (i.e., SV40 enhancer-RSV promoter);
4 corresponding to the replacement of the sequence between PvuII enzyme recognition site and SaCI enzyme recognition site in wild-type 5′LTR with CMV_enhancer-CMV promoter.

3′LTRs were sequentially coded as A, B, C and D, which had corresponding structural features are as follows:
A corresponding to 3′LTR (PvuII-XbaI delete), which was obtained by deleting the promoter/enhancer sequence between PvuII enzyme recognition site and XbaI enzyme recognition site in U3 region;
B corresponding to 3′LTR-SV40 late pA (PvuII-XbaI delete), which was obtained by adding a polyadenylation signal sequence of SV40 (SV40 late pA), as an enhancing element, at the 3′ end of 3′LTR (PvuII-XbaI delete);
C corresponding to 3′LTR (PvuII-SacI delete), which was obtained by deleting the sequences at XbaI enzyme recognition site and between XbaI enzyme recognition site to SacI enzyme recognition site in U3 region of 3′LTR-SV40 late pA (PvuII-XbaI delete);
D corresponding to the 3′LTR-SV40 late pA (PvuII-SacI delete), which was obtained by adding a polyadenylation signal sequence of SV40 (SV40 late pA), as an enhancing element, at the 3′ end of 3′LTR (PvuII-SacI delete).

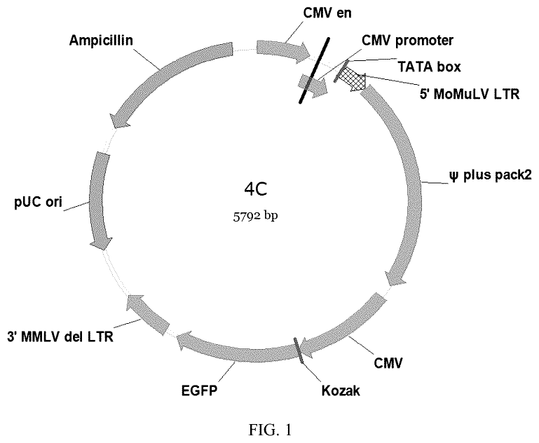

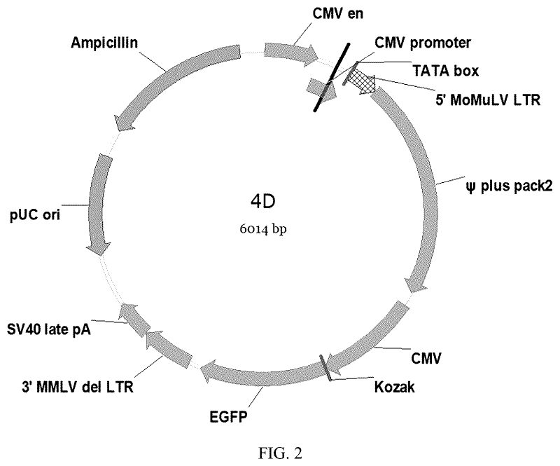

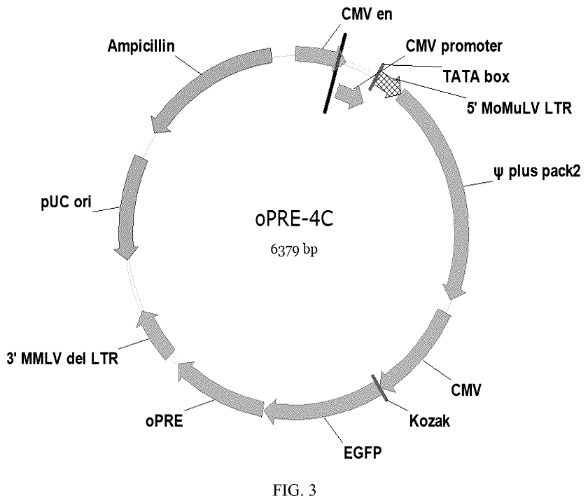

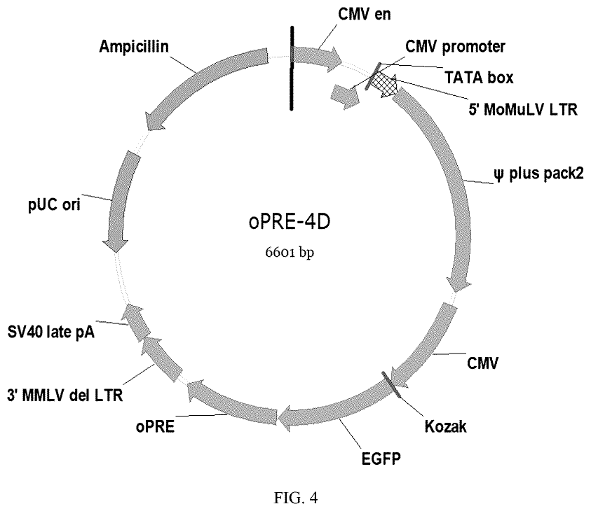

The four 5′LTR structures and four 3′LTR structures were combined to construct 16 different MMLV self-inactivating vectors. Then oPRE element was introduced into the vectors with good test results. A total of 18 MMLV self-inactivating vectors were obtained, which had specific names as shown in Table 1. Schematic diagrams of the structures of MMLV self-inactivating vector 4C, MMLV self-inactivating vector 4D, MMLV self-inactivating vector oPRE-4C and MMLV self-inactivating vector oPRE-4D are shown in FIGS. 1-4.

TABLE 1

| Vector | |
|---|---|
| number | Vector name |
| 1A | pMMLV SIN-CMV > EGFP (PvuII-XbaI delete) |
| 1B | pMMLV SIN > EGFP (PvuII-XbaI delete/SV40pA) |
| 1C | pMMLV SIN-CMV > EGFP (PvuII-SacI delete) |
| 1D | pMMLV SIN-CMV > EGFP (PvuII-SacI delete/SV40pA) |
| 2A | pMMLV SIN-CMV > EGFP (RSV-SacI/PvuII-XbaI delete) |
| 2B | pMMLV SIN-CMV > EGFP (RSV-SacI/PvuII-XbaI delete/SV40pA) |
| 2C | pMMLV SIN-CMV > EGFP (RSV-SacI/PvuII-SacI delete) |
| 2D | pMMLV SIN-CMV > EGFP(RSV-SacI/PvuII-SacI delete/SV40pA) |
| 3A | pMMLV SIN-CMV > EGFP (RSV-SacI/PvuII-SacI delete/SV40pA) |
| 3B | pMMLV SIN-CMV > EGFP (SV40_En-RSV-SacI/PvuII-XbaI delete) |
| 3C | pMMLV SIN-CMV > EGFP (SV40_En-RSV-SacI/PvuII-XbaI delete/SV40pA) |
| 3D | pMMLV SIN-CMV > EGFP (SV40_En-RSV-SacI/PvuII-SacI delete) |
| 4A | pMMLV SIN-CMV > EGFP (CMV_enhancer-CMV promoter-SacI/PvuII-XbaI delete) |
| 4B | pMMLV SIN-CMV > EGFP (CMV_enhancer-CMV promoter-SacI/PvuII-XbaI delete/SV40pA) |
| 4C | pMMLV SIN-CMV > EGFP (CMV_enhancer-CMV promoter-SacI/PvuII-SacI delete) |
| 4D | pMMLV SIN-CMV > EGFP (CMV_enhancer-CMV promoter-SacI/PvuII-SacI delete/SV40pA) |
| oPRE-4C | pMMLV SINCMV > EGFP-oPRE (CMV_enhancer-CMV promoter-SacI/PvuII-SacI delete) |
| oPRE-4D | pMMLV SIN-CMV > EGFP-oPRE (CMV_enhancer-CMV promoter-SacI/PvuII-SacI delete/SV40pA) |

The above 18 vectors can be prepared according to the methods commonly used in the art. The process for constructing 4D vector is taken as an example and described in detail below, and the process for constructing other vectors is similar thereto.

1.1 4D Vector was Constructed Using pMMLV[Exp]-CMV>EGFP with Wild-Type 5′ and 3′LTR as the Backbone Mainly by Two Steps:

1.1.1 The Wild-Type 5′LTR of MMLV was Modified to Construct pMMLV[Exp]-CMV>EGFP (CMV_Enhancer-CMV Promoter-SacI):
(a) pMMLV[Exp]-CMV>EGFP was digested by XmnI+SpeI into fragments with a size of 4.8 kb and 0.9 kb, wherein the small fragment contained the wild-type 5′LTR and the backbone sequences at both ends, and the large backbone fragment of 4.8 kb was recovered.
(b) CMV_enhancer-CMV promoter-partly U3 (PvuII-SacI delete)-R-U5 fragment was subjected to PCR fusion amplification using pMMLV[Exp]-CMV>EGFP as a template. The upstream and downstream primers were added with homologous arms of about 20 bp at the 5′ end for subsequent Gibson cloning reaction, wherein the sequences of the primers are as follows:

XmnI-F:
(SEQ ID NO: 4)
5′-CAGAACTTTAAAAGTGCTCATCATTG-3′;
Backbone-CMV-R:
(SEQ ID NO: 5)
5′-AGTTATGTAACGCGGAATGCAGGTGGCACTTTTCGGG-3′;
Backbone-CMV-F:
(SEQ ID NO: 6)
5′-GTGCCACCTGCATTCCGCGTTACATAACTTACGGTAAA-3′;
SacI-R:
(SEQ ID NO: 7)
5′-GGGCTCTTTTATTGAGCTCTGCTTATATAGACCTCC-3′;
SacI-F:
(SEQ ID NO: 8)
5′-GGTCTATATAAGCAGAGCTCAATAAAAGAGCCCACAAC-3′;
SpeI-R:
(SEQ ID NO: 9)
5′-GCCAGATACAGAGCTAGTTAGC-3′.

(c) The product of PCR amplification of about 1.2 kb in step (b) was recovered.
(d) The product of the digestion of the backbone in step (a) and the PCR fragments obtained in step (c) were added at a certain ratio into Gison system for reaction.
(e) Competent cells were transformed with the reaction product of step (d), which were then spread on LB plate for culture overnight.
(f) Positive clones were identified by PCR and enzyme digestion.
(g) The positive clones with correct sequences were verified by sequencing to obtain pMMLV[Exp]-CMV>EGFP (CMV_enhancer-CMVpromoter-SacI). The sequence of 5′LTR of (CMV_enhancer-CMV promoter-partly U3 (PvuII-SacI delete)-R-U5) is set forth in SEQ ID NO: 1.

1.1.2 The Wild-Type 3′LTR of MMLV was Modified to Construct 4D:
(a) pMMLV[Exp]-CMV>EGFP (CMV_enhancer-CMV promoter-SacI) prepared in 1.1.1 was digested with AfflIII+SalI into fragments with a size of 5.2 kb and 0.8 kb, wherein the small fragment contained the wild-type 3′LTR and the backbone sequences at both ends, and the large backbone fragment of 5.2 kb was recovered.
(b) Partly U3(PvuII-SacI delete)-R-U5-5V40 late pA fragment was subjected to PCR fusion amplification using pMMLV[Exp]-CMV>EGFP as a template. The upstream and downstream primers were added with homologous arms of 20 bp at the 5′ end for subsequent Gibson cloning reaction, wherein the sequences of the primers are as follows:

SalI-F:
(SEQ ID NO: 10)
5′-GAGCGGCCGCCAGCACAGTG-3′;
SacI-R:
(SEQ ID NO: 11)
5′-GGCTCTTTTATTGAGCTCCTGTTCCATCTGTTCCTGACCT-′3;
SacI-F:
(SEQ ID NO: 12)
5′-ACAGATGGAACAGGAGCTCAATAAAAGAGCCCACAAC-3′;
AflIII-R:
(SEQ ID NO: 13)
5′-GCCTTTTGCTGGCCTTTTGCTC-3′.

(c) The product of PCR amplification of about 0.9 kb in step (b) was recovered.
(d) The product of the digestion of the backbone in step (a) and the PCR fragments obtained in step (c) were added at a certain ratio into Gison system for reaction.
(e) Competent cells were transformed with the reaction product of step (d), which were then spread on LB plate for culture overnight.
(f) Positive clones were identified by PCR and enzyme digestion.
(g) The positive clones with correct sequences were verified by sequencing to obtain pMMLV SIN-CMV>EGFP (CMV_enhancer-CMV promoter-SacI/PvuII-SacI del/SV40 pA). The nucleotide sequence of the nucleic acid fragment (partly U3(PvuII-SacI delete)-R-U5-SV40 late pA) formed by the nucleic acid fragment of 3′LTR of pMMLV SIN-CMV>EGFP (CMV_enhancer-CMV promoter-SacI/PvuII-SacI del/SV40 pA) and SV40 late pA is set forth in SEQ ID NO:14.

2. Packaging of Virus and Transduction of HEK293T Cells

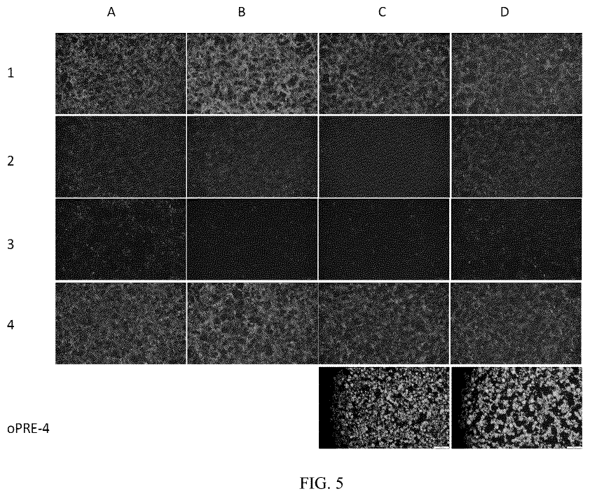

The above 18 vectors were packaged into MMLV virus in HEK293T cells in a 10 cm dish using the calcium phosphate method. After 48 h, the virus supernatant was collected and concentrated with PEG6000. The precipitate obtained from the concentration with PEG was resuspended with 200 μL of MSS. HEK293T cells in 12-well plate were transduced with 100 μL of the resuspended virus solution, and photographed 48 hours after the transduction. The result is shown in FIG. 5.

It can be seen from FIG. 5 that for 5′LTR, the HEK293T cells transduced with the packaged self-inactivating vectors of series 1 with wild-type 5′LTR and the packaged self-inactivating vectors of series 4 with the replacement of the sequence before SacI enzyme recognition site with CMV_enhancer-CMV promoter had similar fluorescence intensity, whereas the series 2 and 3 had very weak fluorescence intensity. It can be seen that, compared with the series 2 and 3, the series 4 had a better 5′LTR structure.

It can be seen from FIG. 5 that for 3′LTR, among series A-D, generally the series B had stronger fluorescence intensity than the series A, and the series D had stronger fluorescence intensity than the series C. It can be seen that the self-inactivating vectors with addition of SV40 late pA behind 3′LTR had stronger fluorescence intensity than the self-inactivating vectors without addition of SV40 late pA behind 3′LTR, indicating that SV40 late pA can effectively enhance the transcription of the viral genome, thereby increasing the virus titer. Therefore, 4D vector is a better MMLV self-inactivating vector, wherein most of the sequences in U3 region of and 3′LTR were deleted, thereby reducing the homology between 5′LTR and 3′LTR, and increasing the stability and biosafety of the vector. Moreover, 4D vector had a viral titer and expression of exogenous genes comparable to that of the wild-type MMLV self-inactivating vector.

Moreover, it can be seen from FIG. 5 that compared with 4C and 4D, oPRE-4C and oPRE-4D had stronger fluorescence intensity, and oPRE-4D had stronger fluorescence intensity than oPRE-4C.

The technical features of the above examples can be combined arbitrarily. To make the description concise, not all possible combinations of the technical features in the above examples are described. However, all the combinations of these technical features should be considered as within the scope of this specification as long as there is no contradiction.

The above examples only represent several embodiments of the present invention, which are described specifically and in detail, but should not be construed as limiting the scope of the patent of the present invention. It should be noted that for those of ordinary skill in the art, several modifications and improvements can be made without departing from the idea of the present invention, and these modifications and improvements all belong to the protection scope of the present invention. Therefore, the protection scope of the patent of the present invention should be subject to the claims.

1. A Moloney murine leukemia virus-based self-inactivating vector, comprising:
5′LTR, wherein a nucleic acid fragment between Pvu II enzyme recognition site and Sac I enzyme recognition site in U3 region of the 5′LTR of the self-inactivating vector is replaced by CMV enhancer and CMV promoter connected to the CMV enhancer;
a target gene or a multiple cloning site for insertion of the target gene; and
3′LTR, wherein a nucleic acid fragment between a PvuII enzyme recognition site and a Sac I enzyme recognition site in U3 region of the 3′LTR of the self-inactivating vector is deleted, and a polyadenylation signal sequence is located at the 3′-terminal of the 3′LTR of the self-inactivating vector.

2. The self-inactivating vector according to claim 1, wherein the nucleotide sequence of the 5′LTR is set forth in SEQ ID NO: 1; and/or, the nucleotide sequence of the 3′LTR is set forth in SEQ ID NO: 2.

3. The self-inactivating vector according to claim 1, wherein the polyadenylation signal sequence is derived from Simian virus 40, and the nucleotide sequence of the polyadenylation signal sequence is set forth in SEQ ID NO: 3.

4. The self-inactivating vector according to claim 1, further comprising a post-transcriptional regulatory element, wherein the post-transcriptional regulatory element is located close to the 5′ terminal of the 3′LTR, and located at the 3′ terminal of the target gene or the multiple cloning site; optionally, the post-transcriptional regulatory element is oPRE.

5. The self-inactivating vector according to claim 1, further comprising a replication origin derived from bacteria and a resistance gene as a marker.

6. The self-inactivating vector according to claim 5, wherein the replication origin is pUC ori;
and the resistance gene is a gene encoding a protein with resistance to a drug selected from the group consisting of kanamycin, zeocin, actinomycin, ampicillin, gentamicin, tetracycline, chloramphenicol, penicillin and a combination thereof.

7. The self-inactivating vector according to claim 1, further comprising a reporter gene.

8. A retrovirus packaging system, comprising at least two plasmids, wherein one of the plasmids is the Moloney murine leukemia virus-based self-inactivating vector according to claim 1, and the self-inactivating vector comprises a target gene.

9. A virus particle, which is generated by transfecting a host cell with the retrovirus packaging system according to claim 8.

10. A gene therapy drug, comprising the virus particle according to claim 9 and a pharmaceutically acceptable excipient.


SEQUENCE LISTING (14 sequences)

SEQ ID NO: 1 - DNA, 688 bp, Artificial Sequence
  Synthetic polynucleotide
tccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 2 - DNA, 303 bp, Artificial Sequence
  Synthetic polynucleotide
aatgaaagaccccacctgtaggtttggcaagctagcttaagtaacgccattttgcaaggcatggaaaaatacataactgagaatagagaagttcagatcaaggtcaggaacagatggaacaggagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 3 - DNA, 222 bp, Artificial Sequence
  Synthetic polynucleotide
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 4 - DNA, 26 bp, Artificial Sequence
  Synthetic polynucleotide
cagaactttaaaagtgctcatcattg

SEQ ID NO: 5 - DNA, 37 bp, Artificial Sequence
  Synthetic polynucleotide
agttatgtaacgcggaatgcaggtggcacttttcggg

SEQ ID NO: 6 - DNA, 38 bp, Artificial Sequence
  Synthetic polynucleotide
gtgccacctgcattccgcgttacataacttacggtaaa

SEQ ID NO: 7 - DNA, 36 bp, Artificial Sequence
  Synthetic polynucleotide
gggctcttttattgagctctgcttatatagacctcc

SEQ ID NO: 8 - DNA, 38 bp, Artificial Sequence
  Synthetic polynucleotide
ggtctatataagcagagctcaataaaagagcccacaac

SEQ ID NO: 9 - DNA, 22 bp, Artificial Sequence
  Synthetic polynucleotide
gccagatacagagctagttagc

SEQ ID NO: 10 - DNA, 20 bp, Artificial Sequence
  Synthetic polynucleotide
gagcggccgccagcacagtg

SEQ ID NO: 11 - DNA, 40 bp, Artificial Sequence
  Synthetic polynucleotide
ggctcttttattgagctcctgttccatctgttcctgacct

SEQ ID NO: 12 - DNA, 37 bp, Artificial Sequence
  Synthetic polynucleotide
acagatggaacaggagctcaataaaagagcccacaac

SEQ ID NO: 13 - DNA, 22 bp, Artificial Sequence
  Synthetic polynucleotide
gccttttgctggccttttgctc

SEQ ID NO: 14 - DNA, 525 bp, Artificial Sequence
  Synthetic polynucleotide
aatgaaagaccccacctgtaggtttggcaagctagcttaagtaacgccattttgcaaggcatggaaaaatacataactgagaatagagaagttcagatcaaggtcaggaacagatggaacaggagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta